(19) United States
(12) Patent Application Publication
(10) Pub. No.: US 2014/0046352 A1
(43) Pub. Date: Feb. 13, 2014

(54) CIRCULAR STAPLER WITH THE CAPABILITY TO RESECT AND ANASTOMOSE VARIABLE VOLUMES OF TISSUE

(71) Applicants: Giuliano Reboa, Hong Kong (CN); Weng Zhiqiang, Suzhou (CN)

(72) Inventors: Giuliano Reboa, Hong Kong (CN); Weng Zhiqiang, Suzhou (CN)

(21) Appl. No.: 13/825,743

(22) PCT Filed: Sep. 22, 2011
(86) PCT No.: PCT/IB11/02753

§ 371 Date: Oct. 17, 2013

Related U.S. Application Data
(60) Provisional application No. 61/385,290, filed on Sep. 22, 2010.

(51) Int. Cl. A61B 17/115; A61B 17/068

(52) U.S. Cl. CPC A61B 17/1155; A61B 17/068
USPC 606/153; 227/175.1

(57) ABSTRACT

According to the disclosure, a variable tissue volume circular stapler instrument (9) may be adapted from those currently used for the removal and treatment of haemorrhoids. An exemplary instrument comprises a handle, a shaft extending from the handle, and a stapling head assembly extending from the shaft. The stapling has a hollow casing that allows a certain amount of tissue to be drawn into the housing via the use of draw string sutures placed in the mucosal layers of the anal wall. The amount of tissue drawn into the housing with current instrumentation may be controlled by providing a variable tissue-volume control mechanism (16) mounted between the casing, the staple pusher mechanism (15) and the central shaft (19) and having fenestrations (18) which correspond to external stapler housing fenestrations (17).

### CROSS-REFERENCE TO RELATED APPLICATIONS

This application claims the benefit of priority of U.S. provisional application No. 61/385,290, of same title, filed on Sep. 22, 2010, the content of which is incorporated herein by reference in its entirety.

### TECHNICAL FIELD

The present disclosure relates to a circular stapler for use in haemorrhoidal surgeries and, more particularly, to a circular stapler having a surgeon-controlled variable tissue volume staple housing.

### BACKGROUND

The modern view of “Haemorrhoidal Disease” and “Obstructed Defecation Syndrome” recognise a common pathogenetic event that is represented by the development, to a different extent, of internal rectal prolapse, in the form of recto-rectal or recto-anal prolapse. Hence, “Haemorroidal Disease” is not merely the consequence of vascular alterations within the rectum but is secondary to this internal rectal prolapse, which becomes the real target of surgical treatment of haemorrhoids. Similar alterations associated with rectal intussusception and rectocele are the base of the onset of “Obstructed Defecation Syndrome”.

On these grounds, the use of a Circular Stapler for the treatment of haemorrhoids was first described by Dr. G. Allegra and presented in his 1990 paper “Particular Experience with Mechanical Sutures: Circular Stapler for Haemorrhoidectomy” which subsequently led to the development of the so called procedure for Prolapsed Haemorrhoids (PPH) presented and developed by Longo et al. and the development of a specially modified Circular Stapler in circa 1998. Since publication the use of a circular stapler for haemorrhoidectomy, or hemorrhoidopexy, using the PPH technique has become widely established.

The subsequent publication of U.S. Pat. No. 6,102,271 (Antonio Longo et al., 15 Aug. 2000), which is incorporated herein by reference, describes a technology and procedure for the surgical removal of haemorrhoids utilising a circular stapling instrument and describes the instrument as having “a stapling end effector having a casing, a hollow stapler driver within the casing, an annular blade having an open distal end is located within the staple driver and an elongated interior drive shaft is reciprocal within the staple driver and the annular blade.” This effectively describes the construction of circular staplers since their conception in the early 1970's. U.S. Pat. No. 6,102,271 goes onto describe the technique whereby tissue is drawn into the staple housing by the placement of circular “purse string” sutures placed within the mucosal layers of the anal canal and the tissue drawn into the stapler housing by pulling said sutures through conduit channels constructed in the staple housing.

This drawing in of anal tissue into the staple housing allows the lifting or “pexy” of the haemorrhoids within the anal passage thus reducing the prolapse of haemorrhoids and interrupting the supply of blood to said haemorrhoids. However, current designs and construction of circular staplers utilised for the procedure for prolapsed haemorrhoids are limited. In fact, a different circular stapler having a particular staple housing volume would need to be used for each anal procedure, depending on the type of anal disorder to be treated.

It may be desirable to provide new innovative design features that will facilitate the use of a single circular stapler for the treatment of a variety of anal disorders that require the resection of larger volumes of tissue in a safe and controlled manner and the subsequent anastomosis. On these grounds, there is the need to develop a circular stapler device including an operator-controlled variable volume staple housing, which may be amenable to the surgical treatment of the different extent of rectal prolapse avoiding multiple procedures and multiple firing of conventional instrumentation, as presently required.

### SUMMARY

The present teachings provide a device and a technique for the simultaneous surgical treatment of the above mentioned conditions by the development of a surgeon-controlled variable tissue volume staple housing facilitating treatment of multiple anal disorders ranger from small anopexy through to large volume, full thickness, bowel resections and anastomosis.

According to various aspects of the disclosure, a variable tissue volume circular stapler instrument may be adapted from those currently used for the removal and treatment of haemorrhoids. An exemplary instrument comprises a handle, a shaft extending from the handle, and a stapling head assembly extending from the shaft. The stapling has a hollow casing that allows a certain amount of tissue to be drawn into the housing via the use of draw string sutures placed in the mucosal layers of the anal wall. The amount of tissue drawn into the housing with current instrumentation may be limited by several factors; most notably by the size of the housing itself (restricting volume of tissue pulled into housing), the internal construction of the housing and the position of a “tissue stop” (restricting proximal movement of tissue secured by the purse string down the central shaft into the staple housing), and also by design of the support column surrounding the central shaft which takes up valuable space.

The present disclosure embodies several new innovative features that will enable a higher volume of tissue to be drawn into the housing whilst maintaining mechanical support of the anvil shaft and other elements critical to the safe operation of the instrument. Further, a stapler in accordance with the disclosure allows the surgeon to control the volume of tissue drawn into the staple housing inter-operatively for subsequent resection and anastomosis of the tissue, thus allowing the surgeon to undertake a much wider range of resections and procedures such as removal of larger recto-anal prolapses and intussusceptions and not restricting the surgeon to haemorrhoid procedures as with currently available circular staplers.

In addition the current embodiment may integrate one or more new and novel design features intended to improve optimal cutting performance of an integral circular cutting blade via the circumferential mounting of the cutting blade as opposed to conventional four point mounting. According to various aspects, staplers according to the disclosure may provide better facilitation of “tissue slide” down the central shaft of circular stapler by lowering of a “tissue stop,” lengthening of a stapler housing to all ingress of additional tissue, enlargement and placement at four points circumferentially around staple housing of fenestrations initially designed to allow passage of suture grabbing instrument for drawing down of tissue but if increased sufficiently will allow tissue to be withdrawn out of fenestration providing additional volume and visualisation of tissue to be resected.

According to various aspects of the disclosure, a circular stapler may facilitate the widening of the application of the instrument to fully endoscopic or laparoscopic assisted endoluminal resection of cancers of the rectum and potentially higher in the colon via intussception of the deseased portion of the colon into the expanded stapler housing for subsequent resection and simultaneous resection.

Some further advantages and embodiments may become evident from the attached drawing.

### BRIEF DESCRIPTION OF THE DRAWING

In the drawing:

FIG. 1 is a side cross-sectional view of a staple housing of a circular stapler according to various aspects of the disclosure.

### DESCRIPTION OF VARIOUS EMBODIMENTS

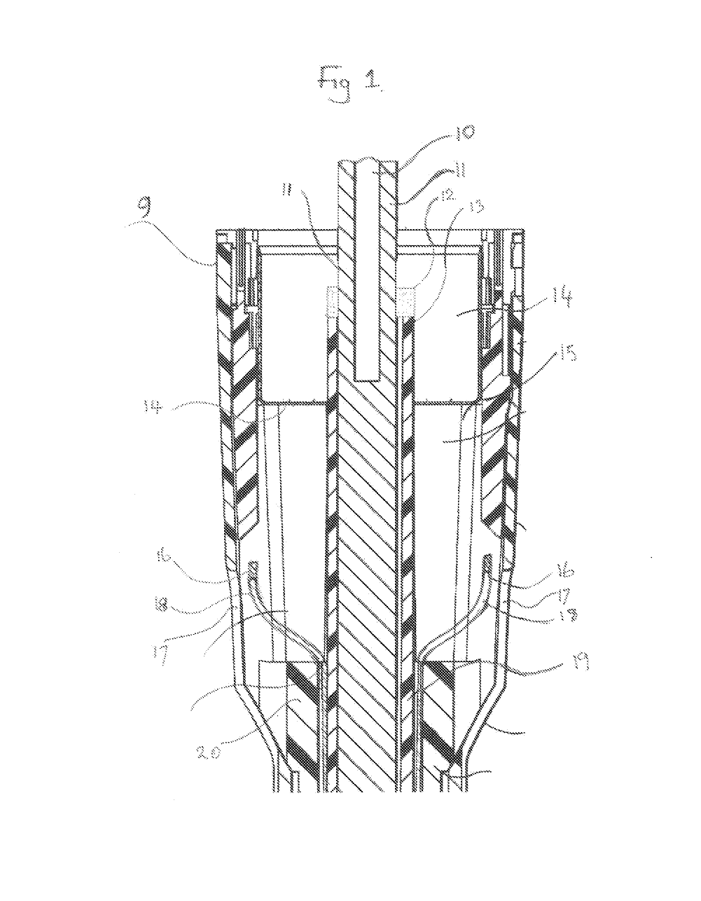

FIG. 1 illustrates the internal construction of a conventional circular stapler 9. The stapler 9 includes an anvil shaft 10 connected to a staple anvil (not shown). The anvil shaft may be driven distally by an anvil rod pusher 11, which is connected to a control knob for closing of device following ingress of tissue (not shown). A “tissue stop” represented by reference numerals 13 has two functions. First, the tissue stop provides additional mechanical support for the anvil rod 10. Second, in the design of conventional circular staplers, the design function of the anvil rod 10 is to prevent movement of tissue into the staple housing following a purse string procedure. This is contrary to the objectives of the design for haemorrhoid, prolapsed, and intussusceptions surgery where smooth movement of tissue into the stapler housing is desirable. In the current design, the tissue stop has been lowered by at least about 2 cm from position 12 to position 13.

A circumferential mounted blade 14 is traditionally mounted internally and supported at four points. This design mounts the circular blade onto a staple pusher mechanism 15 by abutting to a ledge on the staple pusher mechanism 15. The staple pusher mechanism 15 may be a separate moulded component running circumferentially within staple housing. This mechanism offers several advantages in that blade pushing is circumferential and kept more parallel to staple pushing mechanism and more parallel to tissue to be cut.

FIG. 1 also shows the addition of the variable volume control mechanism 16. This is a circular conical shaped element with fenestrations 18 positioned to correspond to external stapler housing fenestrations 17. The variable volume control mechanism 16 is mounted around central shaft 19 and its position may be adjusted distally or proximally by movement of pusher element mounted to surround stapler pusher element 20.

The control for the position of conical element is achieved by rotation of an internal element controlled by a variable slide mechanism (not shown) that facilitates movement of the conical element distally and proximally to predetermined positions within the staple housing to match the variable volume requirements of each procedure or surgeon preference, as would be readily understood by persons skilled in the art.

In the final manufactured embodiment of this concept the external housing may be lengthened to allow possibility of additional tissue intussusceptions.

It will be apparent to those skilled in the art that various modifications and variations can be made to the haemorrhoidal stapler and anal surgery procedures of the present disclosure without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A method for removing internal hemorrhoids from a patient using a circular stapling instrument, said method comprising the steps of:
a) providing a circular stapling instrument having a stapling end effector including:
i) a stapling head assembly having:
a) a substantially hollow casing having an exterior casing surface,
b) a substantially hollow staple driver received in said casing for movement therein between a prefired position and a fired position,
c) an annular blade mounted within said staple driver at a distal end of said staple driver, said annular blade having an open end, and
d) an elongated interior drive shaft reciprocable within said staple driver and said annular blade, and
ii) an anvil assembly having an anvil shaft attached to said elongated interior drive shaft, said anvil assembly movable in response to reciprocation of said interior drive shaft from an open position spaced from said casing to a closed position adjacent to said casing, and when said anvil assembly is in the open position, said interior drive shaft defines a first inner chamber between said staple driver and said shaft, and a second inner chamber between said annular blade and said shaft, said second inner chamber communicating with said first inner chamber and having an open distal end; wherein when said staple driver is in the prefired position, a substantially unobstructed passageway is provided, said passageway extending from said exterior casing surface of said casing through: i) said first inner chamber, ii) said second inner chamber (a), and iii) the open distal end of said second inner chamber;
b) varying the volume of said passageway;
c) placing a suture into the internal hemorrhoids of the patient;
d) inserting said stapling end effector into an anus of the patient with said anvil assembly in the open position;
e) positioning said stapling end effector within the anus of the patient such that said suture and the internal hemorrhoids are located between said stapling head assembly and said anvil assembly;
f) inserting a surgical instrument adapted to grasp said suture into said passageway so as to engage said surgical instrument with said suture;
g) withdrawing said suture through said passageway and out of said casing of said stapling head assembly with said surgical instrument;
h) pulling said suture so as to draw the internal hemorrhoids inwardly around said anvil shaft and said interior drive shaft and into said first and second inner chambers;
i) moving said anvil assembly from the open position spaced from said stapling head assembly to the closed position adjacent to said stapling head assembly so as to clamp the internal hemorrhoids therebetween;
j) firing said circular stapling instrument so as to staple and cut the internal hemorrhoids; and
k) removing said circular stapling instrument from the patient so as to remove the internal hemorrhoids from the patient.

2. The method of claim 1, wherein said varying step comprises slidably moving a variable volume control mechanism distally or proximally relative to the elongated interior drive shaft.

3. A circular stapling instrument adapted for the removal of internal hemorrhoids, said instrument comprising:
a handle;
a shaft extending from said handle;
a stapling head assembly extending from said shaft, said assembly having:
a) a substantially hollow casing having an exterior casing surface,
b) a substantially hollow staple driver received in said casing for movement therein between a prefired position and a fired position,
c) an annular blade mounted within said staple driver at a distal end of said staple driver,
d) an elongated interior drive shaft reciprocable within said staple driver and said annular blade, and
e) a variable volume control mechanism slidably movable relative to the elongated interior drive shaft;
an anvil assembly having an anvil shaft attached to said elongated interior drive shaft, said anvil assembly movable in response to reciprocation of said interior drive shaft from an open position spaced from said casing to a closed position adjacent to said casing, and when said anvil assembly is in the open position, said staple driver and said interior drive shaft define a first inner chamber therebetween, and said annular blade and said interior drive shaft define a second inner chamber therebetween, said second inner chamber communicating with said first inner chamber; and
wherein when said staple driver is in the prefired position, a substantially unobstructed passageway is provided, said passageway extending from said exterior casing surface of said casing into said first and second inner chambers for insertion and withdrawal of a surgical instrument into and from said first and second inner chambers, said passageway being substantially parallel to said interior drive shaft.

4. The circular stapler instrument of claim 3, wherein said variable volume control mechanism may be operably controlled to vary the volume of said passageway.
